(19) United States
(12) Patent Application Publication
(10) Pub. No.: US 2006/0270947 A1
(43) Pub. Date: Nov. 30, 2006

(54) Safety needle assembly

(76) Inventors: Jamieson W. M. Crawford, New York, NY (US); Michael Bennett, Summit, NJ (US); C. Mark Newby, Tuxedo, NY (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/538,992

(22) PCT Filed: Dec. 15, 2003
(86) PCT No.: PCT/US03/39791

§ 371 Date: May 9, 2006

Related U.S. Application Data
(60) Provisional application No. 60/433,760, filed on Dec. 16, 2002.

(51) Int. Cl. A61B 5/00

(52) U.S. Cl. 600/576; 128/919; 604/110

(57) ABSTRACT

A safety needle assembly includes a hub with a needle cannula that has a pointed distal end that projects distally beyond the hub. A shield is hingedly mounted to the hub for rotation from a first position where the needle cannula is exposed to a second position where the needle cannula is shielded. The cannula finger lock has a base end joined integrally to a sidewall of the shield and a free end that projects angularly from the sidewall towards the top wall. Portions of the cannula finger lock near the base end are relatively rigid, whereas portions of the cannula finger lock towards the free end are more flexible.

### FIELD OF THE INVENTION

The present invention relates to a shield for a needle and more particularly to a safety shield assembly that may be used in conjunction with a syringe assembly, a hypodermic needle, a needle assembly, a needle assembly with a needle holder, a blood collection needle, a blood collection set, an intravenous infusion set or other fluid handing devices or assemblies that contain piercing elements.

### BACKGROUND OF THE INVENTION

Disposable medical devices having piercing elements for administering a medication or withdrawing a fluid, such as hypodermic needles, blood collecting needles, fluid handling needles and assemblies thereof, require safe and convenient handling. The piercing elements include, for example, pointed needle cannula or blunt ended cannula.

Safe and convenient handling of disposable medical devices is recognized by those in the medical arts so as to minimize exposure to blood borne pathogens. Safe and convenient handling of disposable medical devices results in the disposal of the medical devices intact.

As a result of this recognition, numerous devices have been developed for shielding needles after use. Many of these devices are somewhat complex and costly. In addition, many of these devices are cumbersome to use in performing procedures. Furthermore, some of the devices are so specific that they preclude use of the device in certain procedures or with certain devices and/or assemblies. For example, some devices employ very short thin needle cannulas. A shield designed to lock near the distal end of one needle cannula might not engage a much shorter needle cannula. Additionally, a shield designed to lock with a wider gauge needle cannula might be more likely to generate a spray upon engaging a much narrower needle cannula. Furthermore, it may be desirable to reduce the force required to effect shielding without reducing the audible and tactile indications of complete shielding.

Therefore, there exists a need for a safety shield assembly: (i) that is manufactured easily; (ii) that is applicable to many devices; (iii) that is simple to use with one hand; (iv) that can be disposed of safely; (v) that does not interfere with normal practices of needle use; (vi) that has tactile features whereby the user may be deterred from contacting the needle, the user may easily orient the needle with the patient and easily actuate and engage the shield assembly; (vii) that has visual features whereby the user may be deterred from contacting the needle, the user may easily orient the needle with the patient and easily actuate and engage the shield assembly; (viii) that is not bulky; (ix) that includes means for minimizing exposure to the user of residual fluid leaking from the needle; and (x) provides minimal exposure to the user because the needle shield is immediately initiated by the user after the needle is withdrawn from the patient's vein.

### SUMMARY OF THE INVENTION

The present invention is a safety shield assembly that comprises: a shield; means for connecting the shield to a fluid handling device that contains a piercing element, such as needle; means for pivoting the shield away from the needle; means for securely covering and/or containing the needle within the shield and means for securely locking the shield in a final non-retractable closed position over the needle.

Preferably, the shield comprises a rearward end, a forward end, a slot or longitudinal opening for housing the used needle in the forward end, means for securing the needle in the slot, means for guiding the needle into the slot, means for connecting the shield and the fluid handling device, means for guiding the user's fingers to move the shield into various positions, and means for retaining the shield securely over the used needle.

Desirably, the means for connecting the shield to the fluid handling device is a collar. Preferably, the shield is connected movably to a collar which is connected to a fluid handling device.

Preferably, the shield is connected to the collar by a hanger bar that engages with a hook arm on the collar so that the shield may be pivoted with respect to the collar into several positions. It is within the purview of the present invention to include any structure for connecting the shield to the collar so that the shield may be pivoted with respect to the collar. These structures include known mechanical hinges and various linkages, living hinges, or combinations of hinges and linkages.

Most preferably, the shield is connected to the collar by an interference fit between the hanger bar and the hook bar. Therefore, the shield always is oriented in a stable position and will not move forward or backwards unless movement of the shield relative to the hanger bar and the hook bar is initiated by the user.

Alternatively, the shield and collar may be a unitary one-piece structure. The one-piece structure may be obtained by many methods, including molding the shield and the collar as a one-piece unit, thereby eliminating the separate shield and collar during the manufacturing assembly process.

The assembly of the present invention may further comprise tactile and visual means for deterring the user from contacting the needle, providing easy orientation of the needle with the patient and providing the user with a guide for actuation and engagement with the shield.

The assembly of the present invention may further comprise means for minimizing exposure by the user to residual fluid leaking from a used needle. For example, a polymer material, such as a gel, may be located in the shield.

Most desirably, the assembly of the present invention is such that the cooperating parts of the assembly provide the means for the shield to move into a forward position over the needle. Thus, by simple movement of the shield into a forward position over the used needle, the assembly is ready for subsequent disposal.

Therefore, the safety shield assembly of the present invention provides minimal exposure of the user to a needle because the shielding is initiated by the user immediately after the needle is withdrawn from the patient's vein.

Desirably, the assembly of the present invention may be used with a syringe assembly, a hypodermic needle, a needle assembly, a needle assembly with a needle holder, a blood collection set, an intravenous infusion set or other fluid handling devices. Preferably, the assembly of the present invention is used with a needle assembly comprising a needle and a hub. Preferably the needle is a conventional double ended needle.

Most preferably, the present invention is used with a needle assembly comprising a hub and a needle connected to the hub whereby the needle comprises a non-patient end and an intravenous end. The collar of the present invention may comprise a hook arm and the shield may be connected movably to the hook arm. Thus the shield may be pivoted with respect to the collar and moved easily into several positions.

Preferably, the collar is fitted non-rotatably with the hub of the needle assembly. Additionally, the collar includes cooperating means that mate with reciprocal means on the shield to help retain the shield in a final closed position, to propel the shield toward the final closed portion and to provide a clear audible and tactile indication of complete shielding. The cooperating means on the collar may include generally chevron-shaped projection formed on a side of the collar substantially diametrically opposite the hook arm or other such structure that provides the hinge connection to the shield. The chevron-shaped structure includes a forward or distal point. Slanting surfaces diverge and extend proximally from the distal point. The slanting surfaces cooperate with the reciprocal means on the shield to generate a deflection of the sidewalls of the shield away from one another. The chevron-shaped structure further includes proximal ends that are convexly arcuate. The convexly arcuate ends of the chevron-shaped structure on the collar cooperate with the reciprocal means on the shield and with the resiliently deflectable sidewalls of the shield to propel the shield toward its closed position, to assist in holding the shield in its closed position and to generate the tactile and audible indication of shielding.

The shield preferably includes a plurality of cannula finger locks spaced axially along the shield for locked engagement with the cannula when the shield is in the final closed position around the needle cannula. Each cannula finger lock preferably projects obliquely from one sidewall of the shield angularly toward the opposed sidewall and the top wall of the shield. Preferably each cannula finger lock has a base that projects rigidly from the respective sidewall and a flexible end that extends from the base. The rigidity of the base and the flexibility of the end may be achieved by relative dimensions along the cannula finger lock. Thus, the base of each cannula finger lock may be relatively thick and/or relatively wide. The end of each cannula finger lock may be tapered in at least one direction to be relatively thin and/or relatively narrow. The rigidity of the base also can be achieved by providing webs extending between the bases of adjacent cannula finger locks, but not extending between the ends of the cannula finger locks. The cannula finger locks are disposed and aligned such that the flexible ends contact the needle cannula when the shield approaches the final closed position. Contact between the cannula and the cannula finger locks will cause the flexible ends of the cannula finger locks to deflect resiliently toward the sidewall from which the cannula finger locks extend. Sufficient rotation of the shield will cause the needle cannula to pass the cannula finger locks. As a result, the flexible end of each cannula finger lock will resiliently return to or toward its undeflected condition for securely trapping the needle cannula in the shield. Forces in a direction to move the shield from the closed position to the open position will cause the needle cannula to engage the top face of each cannula finger lock. The oblique alignment of the cannula finger locks will cause the shield and/or the cannula to deflect slightly so that the cannula slides into engagement with the rigid base of the cannula finger lock. This distinguishes from structures where the entire cannula finger lock is deflectable about its base and/or where the entire cannula finger lock is deflectable at any location along its length.

Preferably, the collar is fitted with the hub of the needle assembly so that the collar cannot rotate around the hub. Additionally, the collar includes cooperating means that mate with reciprocal means on the shield to propel the shield toward a final closed position.

Alternatively, the collar and hub may be a unitary one-piece structure. The one piece structure may be accomplished by many methods including molding the collar and the hub as a one-piece unit thereby eliminating the need to separately assemble the collar to the hub during the manufacturing process.

Most preferably, the collar is fitted with the hub of the needle assembly so that the bevel surface or bevel up surface of the intravenous or distal end of the needle faces the same side of the collar when the shield is in the open position. Alignment of the collar, hub, shield and needle with the bevel surface up makes it easier to insert the needle into the patient without manipulating the assembly. The orientation of the intravenous end of the needle with the bevel up assures the user that the needle is properly oriented properly for use and does not require any manipulation before use. Most notably, the orientation of the shield provides a visual indication to the user of the orientation of the bevel surface of the needle.

Preferably, the shield is capable of pivoting from an open position where the intravenous end of the needle is exposed and bevel up, to an intermediate position where the needle is partially covered, to a final closed nonretractable position where the needle is covered completely and the shield is locked and no longer able to be moved out of the closed position.

Alternatively, it is within the purview of the present invention that the shield, collar and hub is a unitary one-piece structure. The one-piece structure may be accomplished by many methods including molding the shield, collar and hub as a one-piece unit thereby eliminating the need to separately assemble the shield, collar and hub during the manufacturing process.

It is an advantage of the present invention that the shield covering the used intravenous end of the needle provides easy containment of the used needle. A further advantage of the shield is that it will only move upon initiation by the user.

The assembly of the present invention when used with a fluid handling device is also easily disposable when removed from a conventional needle holder, or other such device.

Another important feature of the present invention includes means for locking the shield in a closed permanent position covering the needle. The closed permanent position will generally withstand the normal forces encountered during proper disposal of the safety shield assembly when it is removed from a conventional needle holder.

A notable attribute of the present invention is that it is easily adaptable with many devices. For example, the invention is usable with syringe assemblies, hypodermic needles, needle holders, blood collection needles, blood collection sets, intravenous infusion sets such as catheters or other fluid handling devices or assemblies that contain piercing elements.

Another notable attribute of the present invention is that the tactile and visual features deter the user from touching the needle, allow the user to easily orient the needle with the patient and guide the user to actuate and engage the shield of the assembly.

### BRIEF DESCRIPTION OF THE DRAWINGS

FIG. 1 is a perspective view of the safety shield assembly of the present invention as connected to a needle assembly and related packaging features.

FIG. 2 is a perspective view of the unassembled pieces of FIG. 1.

FIGS. 3A and 3B are bottom views of the shield as shown in FIG. 2.

FIG. 4 is a cross sectional view of the collar as shown in of FIG. 2 taken along lines 4-4 thereof.

FIG. 5 is a cross sectional view of the needle hub as shown in FIG. 2 taken along lines 5-5 thereof.

FIG. 6 is a cross sectional view of the shield of FIG. 2 taken along lines 6-6 thereof.

FIGS. 7-12 illustrate the use of the safety shield assembly with the needle assembly of FIG. 1 with a conventional needle holder.

FIG. 13 is a cross sectional view of the assemblies in use with a conventional needle holder as shown in FIG. 12 taken along lines 13-13 thereof.

FIGS. 14A and 14B are cross-sectional views of the assembly of FIG. 13 taken along lines 14-14 thereof at rotational positions immediately prior to full shielding and immediately after full shielding.

FIGS. 15A and 15B are bottom views of the assembly as shown in FIG. 11.

FIG. 16 illustrates an additional embodiment of the present invention, whereby a gel material is located in the shield as shown in a bottom view of the assemblies of FIG. 11.

FIG. 17 is a perspective view of an additional embodiment of the present invention in use with a blood collection set.

FIG. 18 is a perspective view of an additional embodiment of the present invention in use with a syringe.

FIG. 19 is a perspective view of an additional embodiment of the present invention in use with a catheter.

### DETAILED DESCRIPTION OF THE INVENTION

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

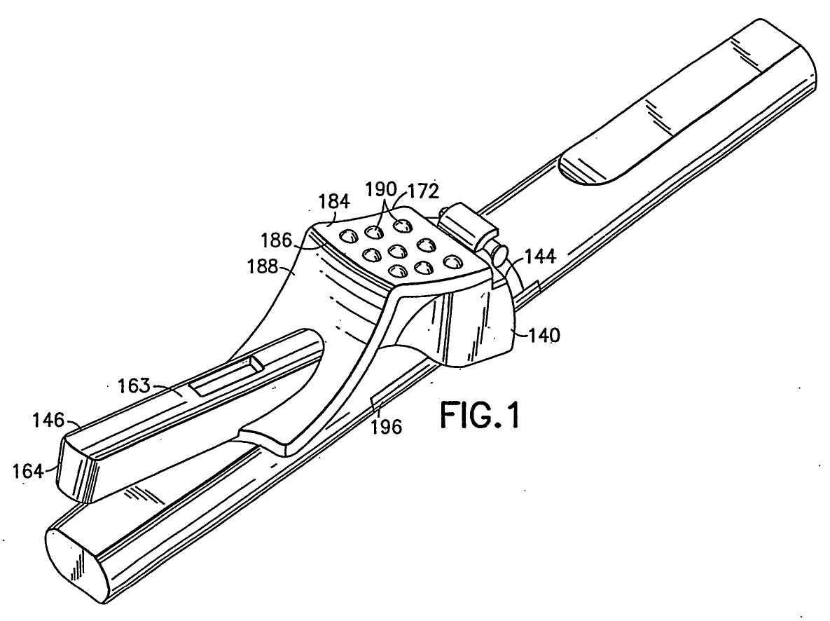

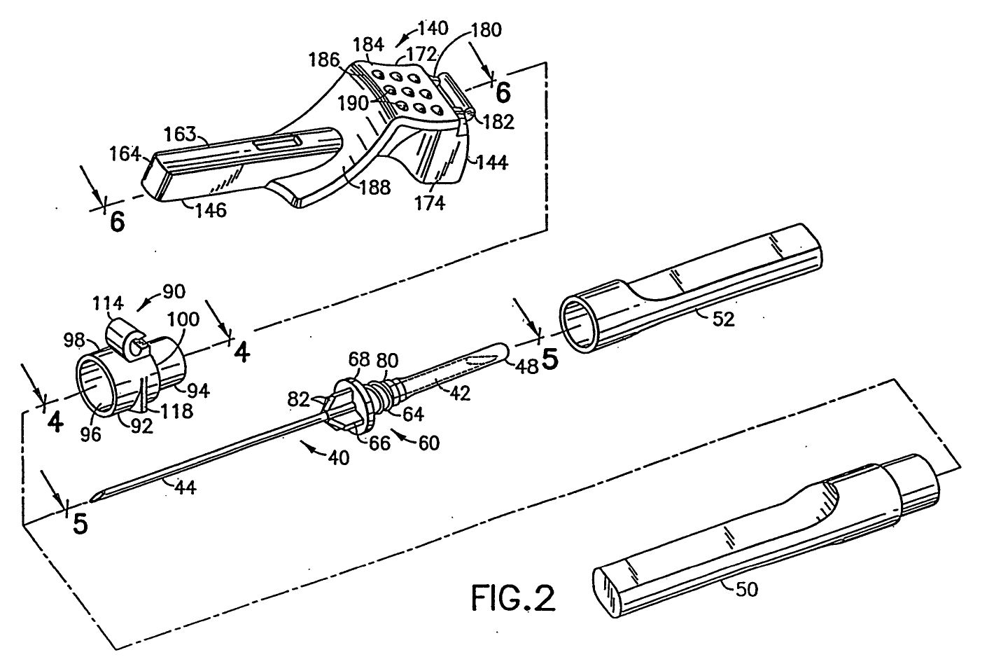

Referring to the drawings in which like reference characters refer to like parts throughout the several views thereof, FIGS. 1 and 2 illustrate a needle assembly with the safety shield assembly of the present invention and the related packaging features. The needle assembly includes a needle 40, a hub 60, packaging features to cover the needle and a label. The safety shield assembly includes a collar 90 and a shield 140.

As shown in FIGS. 2 and 5, needle 40 includes a non-patient end 42, an intravenous end 44 and a passageway 46 extending between the non-patient end and the intravenous end. An elastomeric sleeve 48 covers the non-patient end. A first rigid sleeve 50 covers the intravenous end and a second rigid sleeve 52 covers the non-patient end and the elastomeric sleeve. As shown in FIG. 1, a label 196 may also be applied to the finally assembled parts.

As shown in FIGS. 2 and 5, hub 60 includes a threaded end 64, a ribbed end 66 and passageway 62 extending between the threaded end and the ribbed end. Threaded end 64 and ribbed end 66 are separated by flange 68. Non-patient end 42 of needle 40 extends from threaded end 64 and intravenous end 44 of needle 40 extends from ribbed end 66. Preferably, threaded end 64 comprises male threads 80 for mounting the hub on a conventional needle holder and ribbed end 66 comprises male ribs 82 for connecting the hub and collar 90.

As shown in FIGS. 2 and 4, collar 90 includes a forward skirt 92 and a rearward skirt 94. Forward skirt 92 is cylindrical and comprises an inner circumferential surface 96 and an outer circumferential surface 98. Forward shirt 92 mates with rearward skirt 94 at a shoulder 100. Rearward skirt 94 is cylindrical and comprises an inner circumferential surface 102 and an outer circumferential surface 104 and extends from shoulder 100 opposite of forward skirt 92. The inner diameter of forward skirt 92 is larger than the inner diameter of rearward skirt 94. Alternatively, the inner diameters for collar 90 can be equal. A hook 114 extends from outer circumferential surface 98 of forward skirt 92. Additionally a chevron-shaped protrusion 118 projects outwardly from outer circumferential surface 98 of forward skirt 92 at a side opposite hook 114. The chevron-shape protrusion 118 is substantially symmetrical and has an peak 120 pointed toward forward skirt 92 and ramp surfaces 122 that diverge symmetrically from peak 120 toward rearward skirt 94. Ramp surfaces 122 terminate at rounded ends 124 at the outer side and proximal extremes of chevron-shaped protrusion 118. Rounded ends 124 extend continuously into the proximal side of chevron-shaped protrusion 118 facing toward rearward skirt 94.

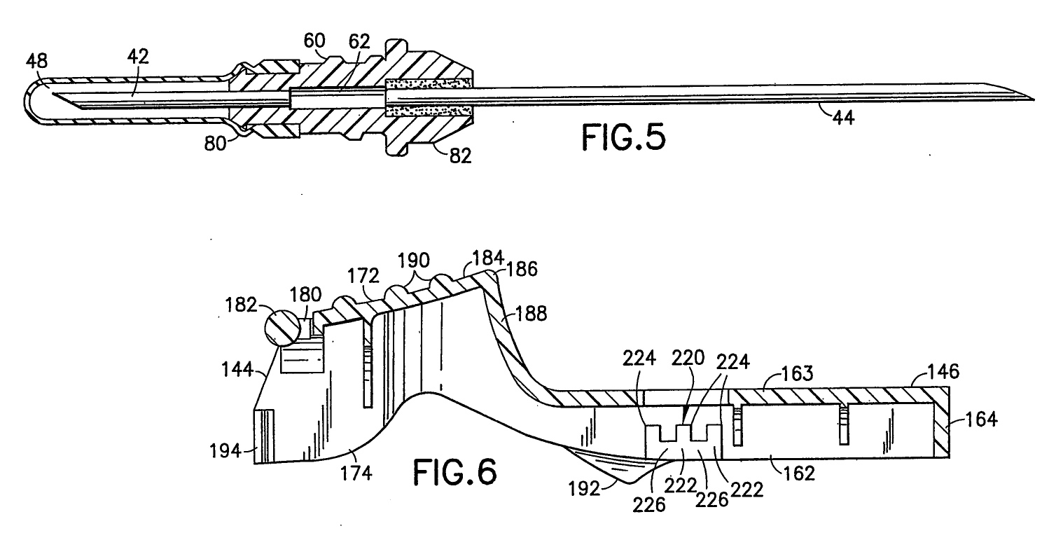

As shown in FIGS. 2 and 6, shield 140 comprises a rearward end 144 and a forward end 146.

Forward end 146 of shield 140 includes a slot or longitudinal opening 160 formed by sidewalls 162 that extend downwardly from top wall 163 and run substantially opposite of one another in parallel along the length of slot 160 towards forward end wall 164. Slot 160 is slightly wider than needle 40. Sidewalls 162 include bottom edges 165 that extend substantially parallel to one another and parallel to top wall 163.

Three cannula finger locks 220 are located at axially spaced locations along one of sidewalls 162 and are configured to secure the used needle. Each cannula finger lock 220 extends from a location on a first of the sidewalls 162 adjacent the bottom edge 165 thereof and projects angularly toward the opposed sidewall 162 and toward the top wall 163, as shown in FIGS. 14A and 14B. The projection of each cannula finger lock 220 from the respective sidewall 162 preferably exceeds half the distance between the respective sidewalls. Each cannula finger lock 220 includes a base 222 that projects unitarily and rigidly from portions of the respective sidewall 167 adjacent bottom edge 165 thereof. Each cannula finger lock 220 further includes an end 224 that extends unitarily and flexibly from base 222 of the respective cannula finger lock. As shown most clearly in FIGS. 14A and 14B, bases 222 are substantially thicker than ends 224 as measured in directions substantially perpendicular to the planes of cannula finger locks 220. Additionally, bases 222 of cannula finger locks are unitarily joined to one another by webs 226. The greater thickness dimensions of bases 222 and the existence of webs 226 substantially prevents bases 222 from flexing in either direction in response to opposed forces between cannula 40 and cannula finger locks 220 during movement of shield 140 in either an opening or closing direction. However, ends 224 of cannula finger locks 220 are deflectable relative to bases 226 as the needle enters slot 160 of shield 140. Once the needle passes the ends of cannula finger locks 220, flexible ends 224 of the cannula finger locks move back to their original positions relative to rigid bases 222 so that the needle is permanently trapped in slot 160 by cannula finger locks 167.

Rearward end 144 of shield 140 defines a collar engaging area 166 that is a continuation of slot 160. Collar engaging area 166 includes a rearward end 168, a forward end 170, a top finger guide area 172, sidewalls 174 that extend downwardly from top finger guide area 172, an underside area 176 dimensioned for surrounding collar 90, and extending arms 180 to support hold hanger bar 182. Sidewalls 174 are spaced apart by a major width adjacent rearward end 168. The major width is selected to enable sidewalls 174 to slide across diametrically opposite side surfaces of forward skirt 92 of collar 90. Sidewalls 174 converge, however, toward forward end 170 to define a minor distance therebetween substantially equal to the distance between sidewalls 162 at forward end 146 of shield 140. Sidewalls 174 include bottom edges 177 that face away from top finger guide area 172. As shown most clearly in FIG. 6, bottom edges 177 curve toward top finger guide area 172 at locations between rearward end 168 and forward end 170 of collar engaging area 166.

The extreme rear ends of sidewalls 174 on collar engaging area 166 include rounded ears 194 that project toward one another from opposed inner surfaces 175 of sidewalls 174. Rounded ears 194 are disposed to engage chevron-shaped projection 118 on collar 90. More particularly, each rounded ear 194 includes a distal surface 195, a proximal surface 197 and a curved surface 198 extending between distal and proximal surfaces 195 and 197. Distal surface 194 is aligned to sidewall 174 at a rake angle of approximately 60° and proximal surface 197 is aligned to sidewall 174 at an angle of approximately 45°. Curved surface 198 extends smoothly and convexly between distal and proximal surfaces 195 and 197. Proximal surfaces 197 of rounded ears 194 win engage ramp surfaces 122 of chevron-shaped projection 118 to deflect sidewalls 174 slightly away from one another as shield 140 approaches the second position. This deflection of sidewalls 174 will occur substantially simultaneously with the deflection of cannula finger locks 220. The apex of curved surface 198 on each rounded ear 194 passes the respective rounded end surface 124 on chevron-shaped projection 118 on collar 90 slightly before cannula finger locks 220 pass the needle cannula. As a result, sidewalls 174 begin to return resiliently toward an undeflected condition. This resilient return of sidewalls 174 cooperates with raked distal surfaces 195 on rounded ears 194 to propel shield 140 into the second position where cannula finger locks 220 pass needle 40. This acceleration of shield 140 caused by the resilient return of sidewalls 174 and raked distal surface 195 of ears 194 also causes sidewalls 174 to snap against chevron-shaped projection 118. This snapping action provides a clear audible and tactile indication of complete shielding and occurs substantially when the used needle is trapped by cannula finger locks 220. The angles of distal and proximal surfaces 195 and 197 of rounded ears 194 affects the performance of shield 140. In particular, a smaller acute angle alignment of proximal face 197 reduces the force required to move shield 140 passed rounded ears 194. A larger acute angle proximal surface 197 of rounded ears 194 requires a greater force to move shield 140 toward the second position. Similarly, the angle between distal surface 195 and sidewall 174 affects the acceleration characteristics as shield 140 is propelled toward the second position in response to the resilient return of sidewalls 174.

Top finger guide area 172 comprises a first ramp 184 that extends slightly on an upwardly slope from the rearward end of the collar engaging area to a shoulder 186. From shoulder 186 extends a second ramp 188 which slopes downwardly towards top section 163. Most preferably, first ramp 184 comprises touch bumps 190. The touch bumps provide a tactile and visual guide to alert the user that the user's finger has contacted the shield and that the shield is in a defined or controlled position. The touch bumps may be any configuration so long as they extend and are distinct from the top finger guide area. The touch bumps may also be of a distinguishing color as compared to the top finger guide area or the shield.

Second ramp 188 has interior surface 192 for urging the needle toward the center of slot 160 as the shield is being rotated into the closed position. The exterior surfaces are slightly inclined and extending radially from the second ramp. The interior surfaces are especially helpful if the longitudinal axis of the needle is misaligned with respect to the longitudinal axis of the hub.

Extending arms 180 are located at rearward end 168 and at the beginning of top finger area 172 and hold hanger bar 182.

The safety shield assembly and the needle assembly are assembled together whereby needle 40 is connected to hub 60 and sealed with adhesive at the ends of the hub. Hub 60 is then joined with collar 90 by ultra-sonic welding techniques or any other bonding techniques, or mechanical fit, whereby rearward annular skirt 94 of collar 90 mates with ribbed end 66 of the hub. Male ribs 82 of the hub are contained or forced fitted within inner sidewall 102 of rearward annular skirt 94 of collar 90. The collar is aligned with the intravenous end of the needle whereby the hook arm is aligned with the bevel up of the needle. Then rigid sleeve 50 is force fitted into inner side wall 96 of forward skirt 92 of collar 90 to cover the needle. Thereafter, shield 140 is connected to collar 90 whereby hanger bar 182 is force fitted into hook member 114 whereby slot 160 faces rigid sleeve 50. Most preferably, the shield is connected to the collar by a force fit or interface fit between the hanger bar and the hook bar. Therefore, the shield is always oriented in a stable position and will not move unless movement of the shield is positively initiated by the user. To assemble the last piece, shield 140 is moved towards rigid sleeve 50 and second rigid sleeve 52 is force fitted onto outer sidewall 104 of rearward skirt 94 of collar 90.

In addition, a label 196 may be applied to the finally assembled parts. The label may be used to prevent tamper resistance of the parts, so that they are not reused.

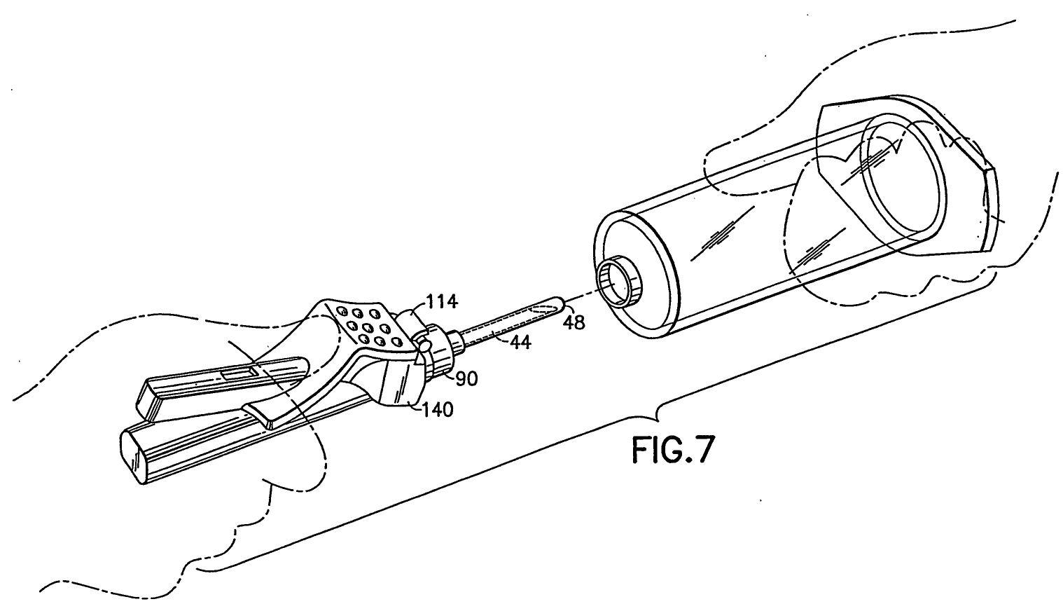

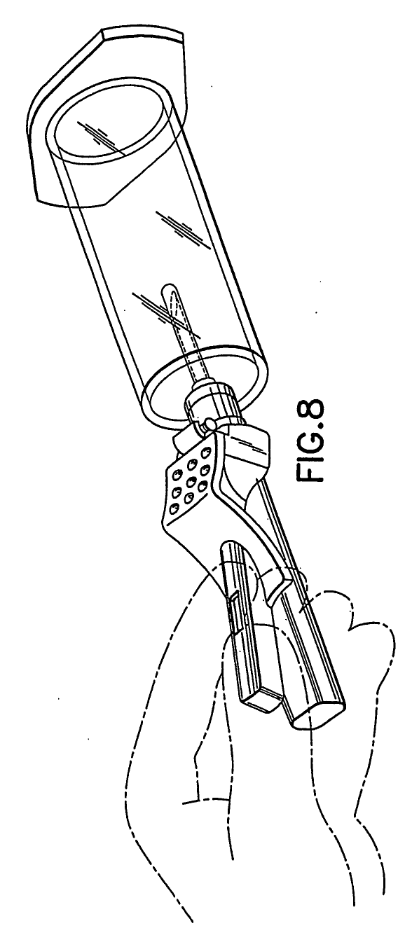

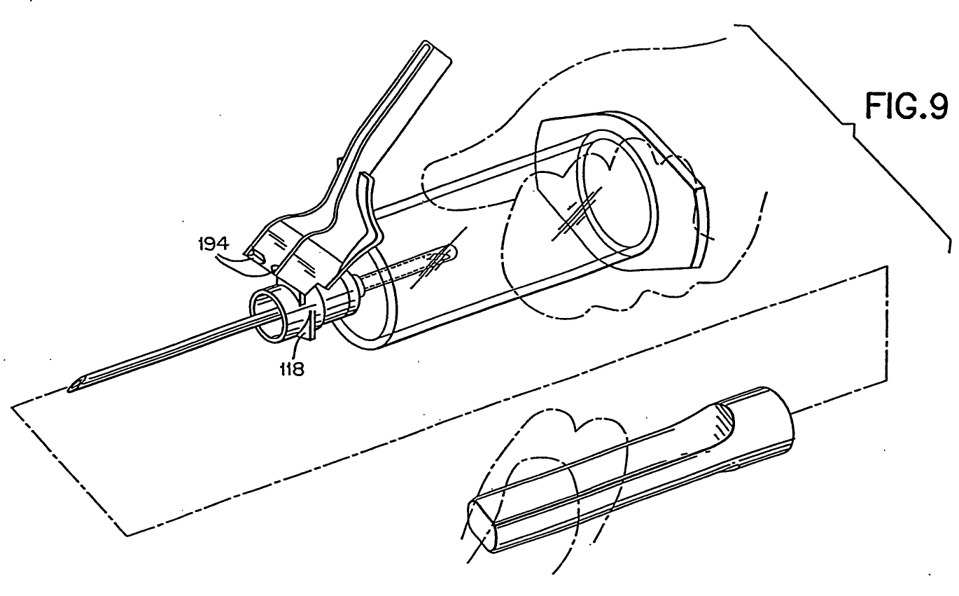

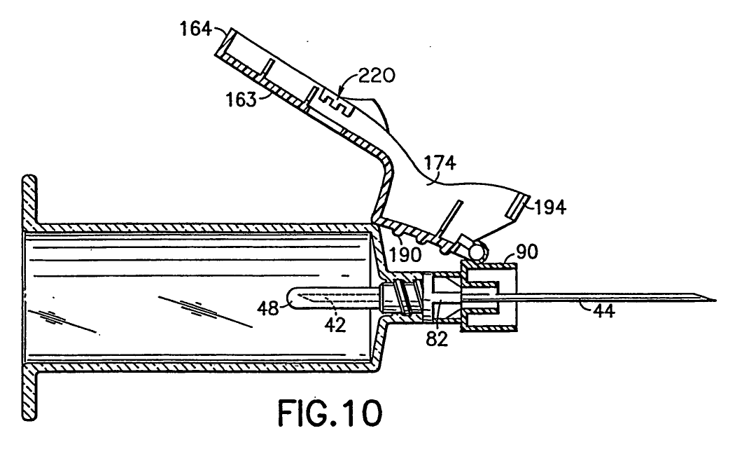

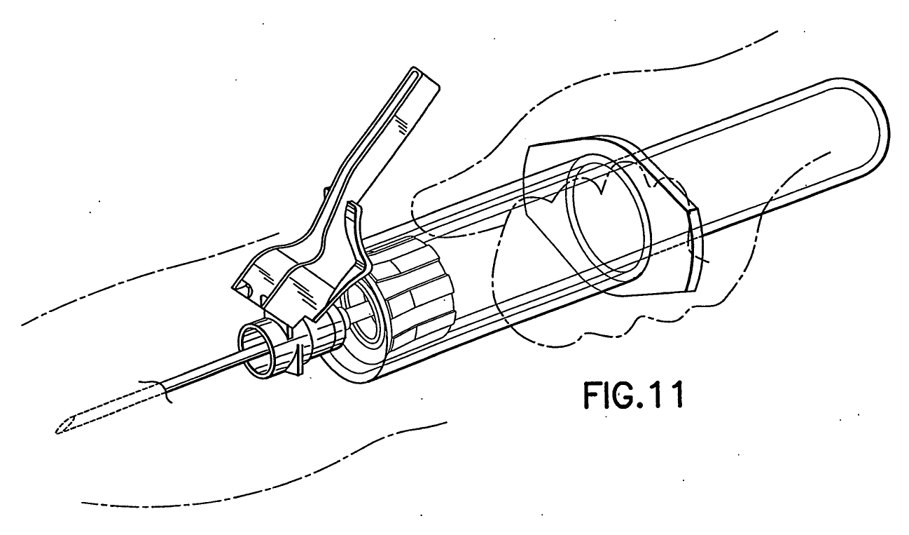

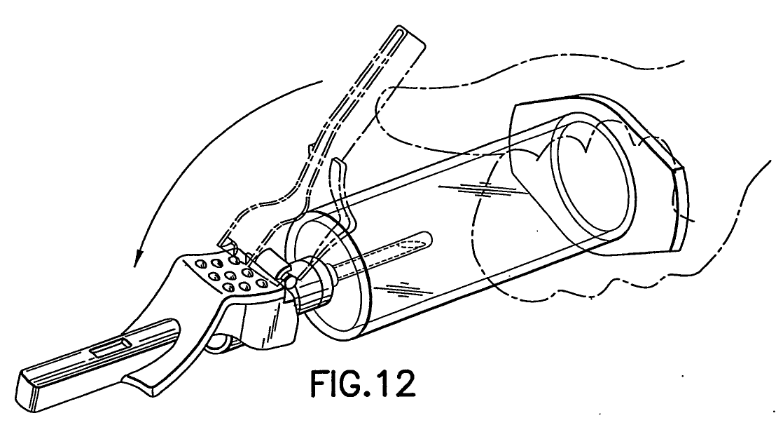

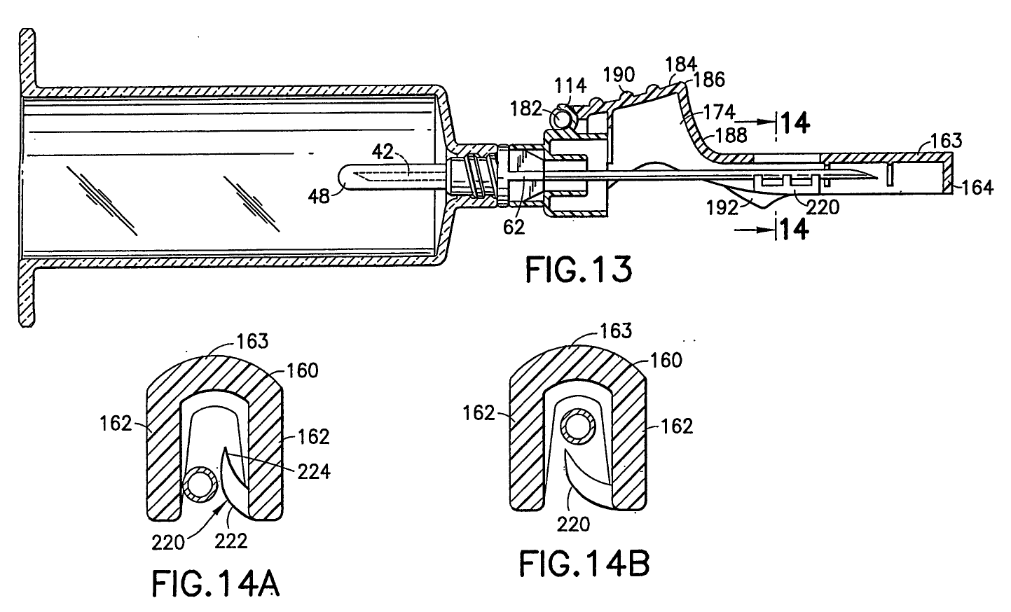

In use, as shown in FIGS. 7-15, the non-patient needle shield is removed and then a needle holder is screwed onto the hub of the needle. As specifically shown in FIGS. 8 and 12 the shield is then rotated back by the user towards the needle holder. Then as shown in FIG. 9, the intravenous needle shield is removed from covering the intravenous needle. Then as shown in FIG. 10, a venipuncture is conducted whereby the intravenous end of the needle is inserted into a vein of a patient and an evacuated tube having a closure is inserted into the needle holder. Then as shown in FIGS. 11 and 13, when the venipuncture is complete the user easily rotates the shield from the open position towards the intravenous needle to an intermediate position and then the user pushes on the shield at the top finger guide area to move the shield into a final, non-retractable locked position whereby the needle is trapped in the longitudinal opening. More particularly, needle 40 contacts flexible ends 224 of cannula finger locks 220. The engagement of needle 40 with cannula finger locks 220 causes flexible ends 224 of cannula finger locks 220 to deflect relative to rigid bases 222 toward top wall 163 and toward sidewall 162 from which cannula finger locks 220 project. Sufficient rotation of shield 140 will cause needle 40 to pass cannula finger locks 220. As a result, flexible ends 224 of all three cannula finger locks 220 will return resiliently to an undeflected condition relative to rigid bases 222. Thus, needle 40 will be trapped above cannula finger locks 220. The angular alignment and relative dimensions of each cannula finger lock 220 provides good resistance to forces that tend to move shield 140 back toward the open position. More particularly, forces in an opening direction of shield 140 will cause engagement between needle 40 and the upper surface of flexible ends 224 of cannula finger locks 220. The angular alignment of cannula finger locks 220 will cause slight deflection of needle 40 and shield 140 that will cause relative sliding movement between needle 40 and cannula finger locks 220. As a result, needle 40 will slide into contact with rigid base 222 and web 226. Hence, shield 140 cannot be returned to the opened condition.

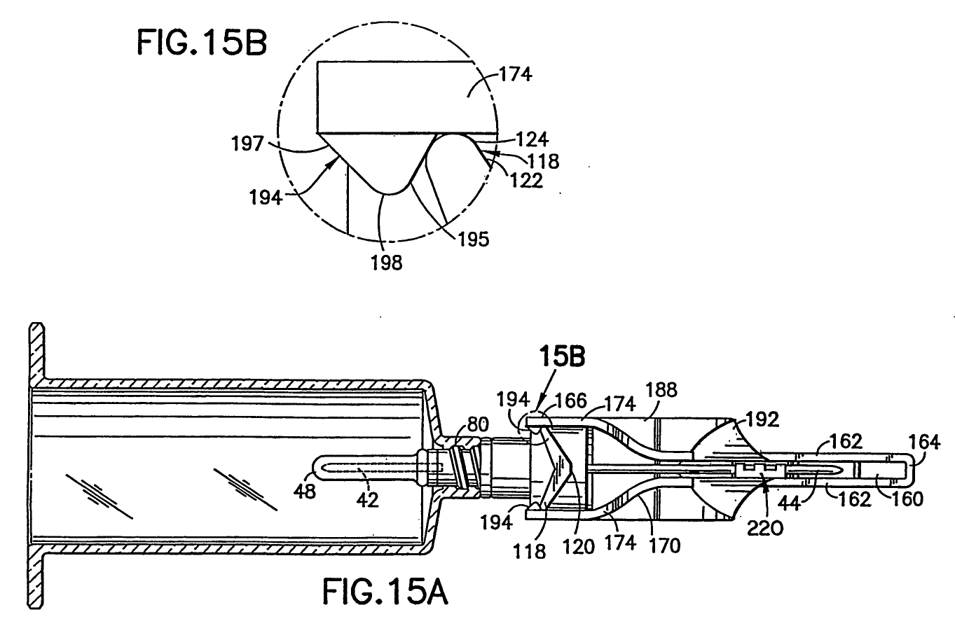

Needle 40 is contained within shield 140 as the shield is pivoted into the closed position. More particularly, proximal surfaces 197 of rounded ears 194 move over ramp surfaces 122 of chevron-shaped projection 118 and cause sidewalls 174 to deflect away from one another. The angularly aligned proximal faces 197 of rounded ears 194 ensures easy movement of shield 140. Additionally, the resiliency of sidewalls 174 and the angular alignment of distal surface 195 of ears 194 causes a cooperation with rounded proximal ends 124 of chevron-shaped projection 118 to accelerate shield 140 into the full shielding closed position. Thus needle 40 snaps past cannula finger locks 220 and is trapped as shown in FIGS. 14 and 15 to ensure complete locking of shield 140 in the closed position. This accelerated movement of shield 140 helps to generate a clear audible and tactile indication of complete shielding.

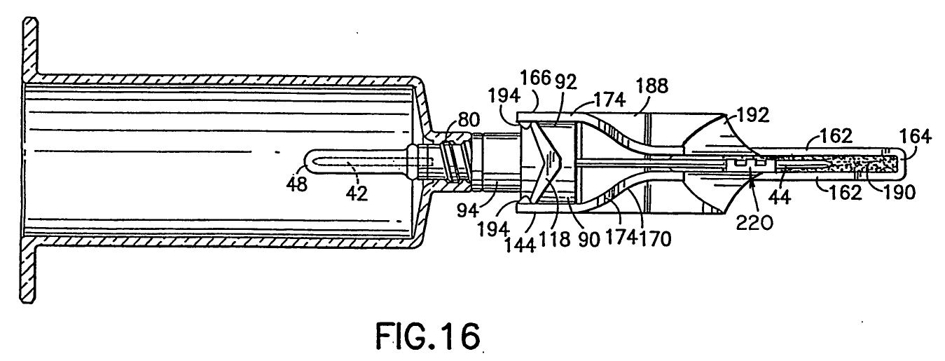

Alternatively as shown in FIG. 16, a gel material 190 is located in shield 140 so that when the needle snaps past cannula finger locks 220 it will come to rest in gel material 190. The gel material will contain any residual fluid that may be on the needle. Simultaneously, rounded ears or projections 198 move over rounded proximal ends 124 of chevron-shaped projection 118. This causes sidewalls 174 to deflect away from one another and then to snap back into engagement with collar 90 to provide a clear audible and tactile indication of complete shielding.

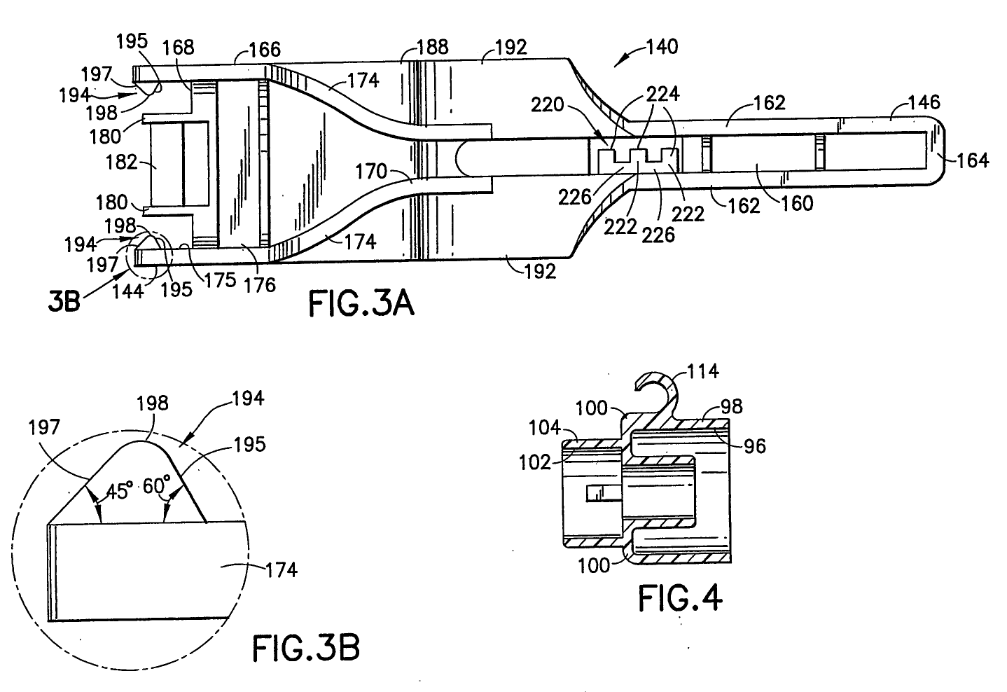

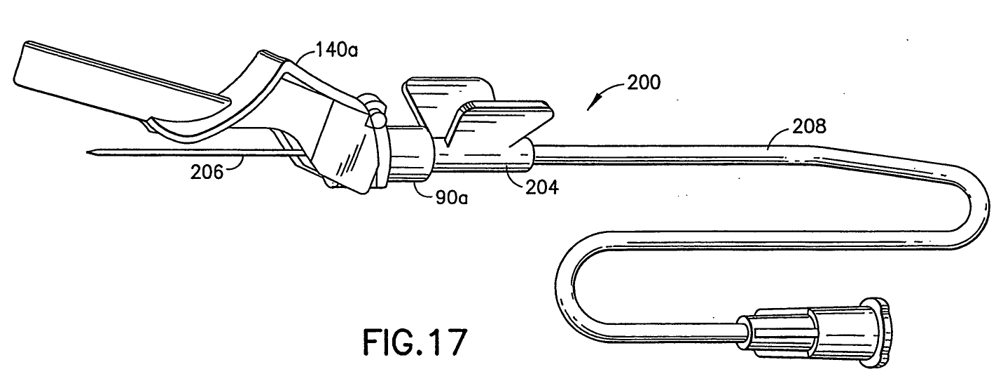

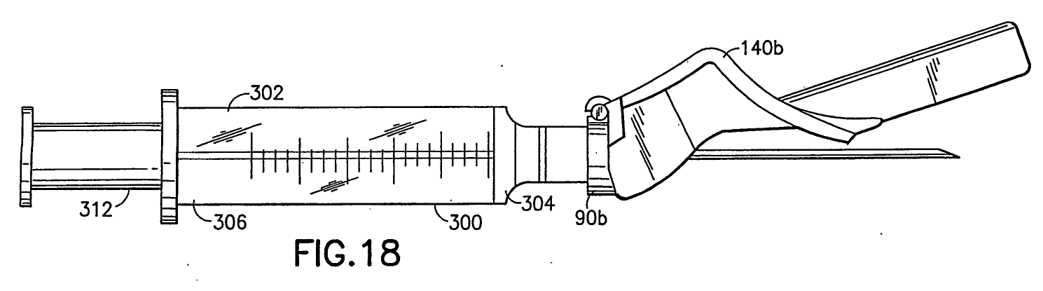

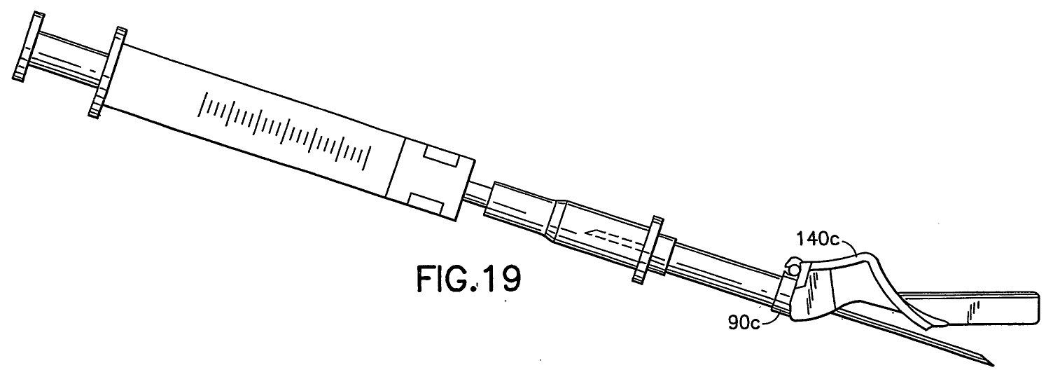

FIGS. 17, 18, and 19 are further embodiments of the invention that include may components which are substantially identical to the components. FIGS. 1-3. Accordingly, similar components performing similar functions will be numbered identically to those components of FIGS. 1-3, except that a suffix “a” will be used to identify those similar components in FIG. 17, a suffix “b” will be used to identify those similar components in FIG. 18 and a suffix “c” will be used to identify those similar components in FIG. 19.

Alternatively, the safety shield assembly of the present invention may be used in conjunction with a conventional intravenous (IV) fusion set, as illustrated in FIG. 17.

For purposes of illustration, shield 140a and collar 90a are connected to a conventional IV infusion set, 200, or butterfly structure comprising a needle body with a needle hub 204 extending from the forward end of the needle body and a needle 206 embedded in hub 204. Extending from the rearward end of the needle body is flexible tubing 208 which is conventional and utilized to allow the user to manipulate the structure and to connect it subsequently to supplies of infusion liquids or for the return of collected blood if the arrangement is being used to collect blood.

Infusion set 200 further comprises flexible wings 210 attached to and projecting outwardly from needle hub 204.

Alternatively, the safety shield assembly of the present invention may be used in conjunction with a syringe, as illustrated in FIG. 18.

For purposes of illustration, shield 140b and collar 90b are connected to a conventional hypodermic syringe 300 comprising a syringe barrel 302 having a distal end 304 a proximal end 306 and a plunger 312.

Alternatively, the present invention may be used in conjunction with a catheter as illustrated in FIG. 19.

The shield and collar of the safety shield assembly of the present invention are comprised of moldable parts which can be mass produced from a variety of materials including, for example, polyethylene, polyvinyl chloride, polystyrene or polyethylene and the like. Materials will be selected which will provide the proper covering and support for the structure of the invention in its use, but which will provide also a degree of resiliency for the purpose of providing the cooperative movement relative to the shield and the collar of the assembly.

What is claimed is:

1. A safety needle assembly comprising a needle hub with proximal and distal ends and a passage extending between said ends, a needle cannula mounted to said passage of said needle hub and having a pointed distal end projecting beyond said distal end of said hub, a shield having proximal and distal ends, said proximal end of said shield being hingedly mounted to said hub for rotation from a first position where said shield is spaced from said needle cannula to a second position where said shield substantially surrounds said needle cannula, said shield comprising a top wall and opposed first and second sidewalls extending from said top wall, said sidewalls having edges remote from said top wall, at least one cannula finger lock having a base end integral with the first sidewall and a free end projecting angularly towards said top wall, portions of said cannula finger lock in proximity to said base end thereof being substantially rigid, and portions of said cannula finger lock in proximity to said free end thereof being resiliently deflectable, whereby resiliently deflectable portions of said cannula finger lock yield as said shield is rotated into said second position to permit said needle cannula to be trapped between said cannula finger lock and said top wall, and whereby substantially rigid portions of said cannula finger lock prevent re-exposure of said needle cannula after said shield is in said second position.

2. The safety needle assembly of claim 1, wherein said portions of said cannula finger lock in proximity to said free end are made more flexible than portions of said cannula finger lock near said base end by forming said cannula finger lock with reduced dimensions at locations in proximity to said free end thereof.

3. The safety needle assembly of claim 2, wherein portions of said cannula finger lock in proximity to said free end thereof are resiliently deflectable in directions transverse to said needle cannula when said shield is rotated into said second position, said portions of said needle cannula in proximity to said free end being of reduced dimension in directions extending substantially along a direction of deformation of said cannula finger lock.

4. The safety needle assembly of claim 2, wherein portions of said cannula finger lock in proximity to said free end thereof are resiliently deflectable in directions transverse to said needle cannula when said shield is rotated into said second position, said portions of said needle cannula in proximity to said free end being of reduced dimension in directions substantially parallel to said needle cannula when said shield is in said second position.

5. The safety needle assembly of claim 1, wherein said at least one cannula finger lock comprises at least first and second cannula finger locks, a web extending between portions of said first and second cannula finger lock substantially adjacent the respective base ends thereof for making said cannula finger lock substantially rigid at locations in proximity to said base ends thereof.

6. The safety needle assembly of claim 1, wherein said at least one cannula finger lock comprises a plurality of cannula finger lock, portions of the respective cannula finger locks in proximity to the base ends thereof being connected to one another by webs, portions of said cannula finger locks adjacent the free ends thereof being substantially free of said webs.

7. The safety needle assembly of claim 1, wherein said cannula finger lock is formed unitarily with said shield.

8. The safety needle assembly of claim 1, wherein said needle cannula has a proximal end projecting proximally beyond said hub.

9. The safety needle assembly of claim 1, wherein said hub further includes at least one detent formed thereon, and wherein said shield includes an ear formed thereon and configured for engaging said detent on said hub when said shield is in said second position, said detent and said ear being configured for providing audible and tactile indication of said shield reaching said second position.

10. A safety needle assembly comprising a medical device, a needle cannula mounted to the medical device and having a pointed distal end projecting beyond said medical device, a shield having an elongate slot, said slot being dimensioned for receiving at least said pointed distal end of said needle cannula, said shield being hingedly mounted in proximity to said medical device for rotation from a first position where said needle cannula is exposed to a second position where at least said pointed distal end of said needle cannula is in said slot of said shield, at least one cannula finger lock having a substantially rigid base end projecting from said shield and partly into said slot, said cannula finger lock further having a resiliently deflectable free end, whereby said resiliently deflectable free end of said cannula finger lock yields as said shield is rotated into said second position to permit said needle cannula to be trapped in said shield by said cannula finger lock, and whereby said substantially rigid base end of said cannula finger lock prevents re-exposure of said needle cannula after said shield is in said second position.

11. The safety needle assembly of claim 10, wherein said portions of said cannula finger lock in proximity to said free end are made more flexible than portions of said cannula finger lock near said base end by forming said cannula finger lock with reduced dimensions at locations in proximity to said free end thereof.

12. The safety needle assembly of claim 11, wherein portions of said cannula finger lock in proximity to said free end thereof are resiliently deflectable in directions transverse to said needle cannula when said shield is rotated into said second position, said portions of said needle cannula in proximity to said free end being of reduced dimension in directions extending substantially along a direction of deformation of said cannula finger lock.

13. The safety needle assembly of claim 11, wherein portions of said cannula finger lock in proximity to said free end thereof are resiliently deflectable in directions transverse to said needle cannula when said shield is rotated into said second position, said portions of said needle cannula in proximity to said free end being of reduced dimension in directions substantially parallel to said needle cannula when said shield is in said second position.

14. The safety needle assembly of claim 10, wherein said at least one cannula finger lock comprises at least first and second cannula finger locks, a web extending between portions of said first and second cannula finger lock substantially adjacent the respective base ends thereof for making said cannula finger lock substantially rigid at locations in proximity to said base ends thereof.

15. The safety needle assembly of claim 10, wherein said at least one cannula finger lock comprises a plurality of cannula finger lock, portions of the respective cannula finger locks in proximity to the base ends thereof being connected to one another by webs, portions of said cannula finger locks adjacent the free ends thereof being substantially free of said webs.

16. The safety needle assembly of claim 10, wherein said cannula finger lock is formed unitarily with said shield.
